(19) United States
(12) Patent Application Publication
(10) Pub. No.: US 2021/0395838 A1
(43) Pub. Date: Dec. 23, 2021

(54) COMPOSITIONS AND METHODS RELATED TO REPORTER SYSTEMS AND LARGE ANIMAL MODELS FOR EVALUATING GENE EDITING TECHNOLOGY

(71) Applicant: North Carolina State University, Raleigh, NC (US)

(72) Inventors: Jorge A. Piedrahita, Raleigh, NC (US); Kathryn Polkoff, Raleigh, NC (US)

(21) Appl. No.: 17/421,279

(22) PCT Filed: Jan. 10, 2020
(86) PCT No.: PCT/US20/13085

§ 371 Date: Jul. 7, 2021

Related U.S. Application Data
(60) Provisional application No. 62/791,440, filed on Jan. 11, 2019.

(51) Int. Cl. C12Q 1/6897; A61K 49/00; A01K 67/027

(52) U.S. Cl. CPC C12Q 1/6897; A61K 49/0008; A01K 2267/0393; A01K 2227/108; A01K 67/0275

(57) ABSTRACT

The present disclosure provides compositions and methods related to the assessment of gene editing technologies in an animal model with single-cell resolution. In particular, the present disclosure provides a novel gene editing reporter system and transgenic animal platform for testing and optimizing gene editing technologies in vivo prior to implementation in humans.

### CROSS REFERENCE TO RELATED APPLICATION

This application claims priority to and the benefit of U.S. Provisional Patent Application No. 62/791,440 filed Jan. 11, 2018, which is incorporated herein by reference in its entirety for all purposes.

### FIELD

The present disclosure provides compositions and methods related to the assessment of gene editing technologies in an animal model with single-cell resolution. In particular, the present disclosure provides a novel gene editing reporter system and transgenic animal platform for testing and optimizing gene editing technologies in vivo prior to implementation in humans.

### BACKGROUND

Clinical applications of CRISPR-CAS or other gene editing technologies are generally regarded as the future of treatment and correction of genetic disorders in humans. For these technologies to be implemented safely, it is critical that the effectiveness of the systems (on-target effects) as well as their side effects (off-target effects and immune response) are thoroughly characterized. Similarly, new and improved in vivo gene delivery systems will need to be scaled-up and tested before human applications are possible. At present, there is no animal model with a physiology and size similar to humans that can be used to detect the on- and off-target cleavage efficiency and/or tissue or cell type specificity of gene editors at a single cell resolution. With gene editing enzymes being continuously being developed and improved, and novel delivery methods emerging, there is a growing need for a cost-effective large animal reporter system that can be used to develop safety data before human clinical trials.

### SUMMARY

Embodiments of the present disclosure include a nucleic acid reporter construct for evaluating functionality of a gene editing system. In accordance with these embodiments, the construct includes a first reporter cassette comprising: a first in-frame non-functional fluorescent reporter comprising at least one self-tolerizing peptide and at least one unknown gene editing target site; a known gene editing target site from at least one human gene; and a first out-of-frame functional fluorescent reporter. The construct also includes a second reporter cassette comprising: a base editor region comprising at least one base editor target site; a second in-frame non-functional fluorescent reporter comprising at least one self-tolerizing peptide; an off-target array region comprising a known gene editing target site from at least one human gene; and a second out-of-frame functional fluorescent reporter. In accordance with these embodiments, the first and second reporter cassettes detect efficiency of a gene editing system based on fluorescence of the at least one first or second out-of-frame functional fluorescent reporter.

In some embodiments, the first or second in-frame non-functional fluorescent reporter is GFP (e.g., H2B-GFP), mCherry (e.g., H2B-mCherry), or BFP (e.g., H2B-BFP). In some embodiments, the at least one self-tolerizing peptide comprises an antigenic peptide from a GFP fluorescent reporter, an mCherry fluorescent reporter, or a BFP fluorescent reporter. In some embodiments, the at least one unknown gene editing target site comprises a putative PAM sequence. In some embodiments, the putative PAM sequence comprises one or more of NGG, NAG, NGGAG, and TTTN. In some embodiments, the known gene editing target site from at least one human gene comprises at least one CRISPR target site from a FANCF gene, a VEGFA gene, a HEK site (e.g., a HEK1 intronic site 1, a HEK3 site, a HEK4 site), an EMX gene, or an RNF gene.

In some embodiments, the known gene editing target site from at least one human gene comprises a plurality of on-target and off-target gene editor target sites. In some embodiments, the known gene editing target site from at least one human gene comprises at least one binding site for a CRISPR associated protein. In some embodiments, the first or second out-of-frame functional fluorescent reporter is GFP (e.g., H2B-GFP), mCherry (e.g., H2B-mCherry), or BFP (e.g., H2B-BFP). In some embodiments, the first or second out-of-frame functional fluorescent reporter is nuclear localized. In some embodiments, the first or second out-of-frame functional fluorescent reporter comprises a 2A peptide sequence. In some embodiments, the at least one base editor target site in the base editor region comprises at least one of an adenine base editor (ABE) or a cytosine base editor (CBE).

In some embodiments, editing of the at least one base editor target site produces a new proximal ATG site and allows for expression of the second out-of-frame functional fluorescent reporter. In some embodiments, the known gene editing target site from the at least one human gene in the off-target array region comprises at least one CRISPR target site from a FANCF gene, a VEGFA gene, a HEK site (e.g., a HEK1 intronic site 1, a HEK3 site, a HEK4 site), an EMX gene, or an RNF gene. In some embodiments, the known gene editing target site from the at least one human gene in the off-target array region comprises a plurality of on-target and off-target gene editor target sites. In some embodiments, the known gene editing target site from the at least one human gene in the off-target array region comprises at least one binding site for a gene editor associated protein.

Embodiments of the present disclosure also include a cell comprising the reporter construct described above. In some embodiments, the cell is one or more of a human cell, a primate cell, a porcine cell, a murine cell, a mammalian cell, an insect cell, an amphibian cell, an avian cell, or a fish cell.

Embodiments of the present disclosure also include a transgenic organism comprising the reporter construct described above. In some embodiments, the transgenic organism is porcine.

Embodiments of the present disclosure also include a method of assessing functionality of a gene editing system. In accordance with these embodiments, the method includes subjecting a transgenic organism comprising the reporter construct described above to a gene editing system and detecting fluorescence of the at least one first and/or second out-of-frame functional fluorescent reporter.

Embodiments of the present disclosure also include a nucleic acid reporter construct for evaluating functionality of a gene editing system. In accordance with these embodiments, the construct includes a reporter cassette comprising: a base editor region comprising at least one base editor target site; an in-frame non-functional fluorescent reporter comprising at least one self-tolerizing peptide; and an out-of-frame functional fluorescent reporter. In accordance with these embodiments, the reporter cassette detects efficiency of a gene editing system based on fluorescence of the at least one out-of-frame functional fluorescent reporter.

In some embodiments, the in-frame non-functional fluorescent reporter is GFP, mCherry, or BFP. In some embodiments, the at least one self-tolerizing peptide comprises an antigenic peptide from a GFP fluorescent reporter, an mCherry fluorescent reporter, or a BFP fluorescent reporter. In some embodiments, the out-of-frame functional fluorescent reporter is nuclear localized. In some embodiments, the at least one base editor target site in the base editor region comprises at least one of an adenine base editor (ABE) or a cytosine base editor (CBE).

In some embodiments, editing of the at least one base editor target site produces a new proximal ATG site and allows for expression of the second out-of-frame functional fluorescent reporter.

Embodiments of the present disclosure also include a nucleic acid reporter construct for evaluating functionality of a gene delivery system. In accordance with these embodiments, the construct includes a first reporter cassette comprising: a first in-frame non-functional fluorescent reporter comprising at least one self-tolerizing peptide and at least one unknown gene editing target site; a known gene editing target site from at least one human gene; and a first out-of-frame functional fluorescent reporter. The construct also includes a second reporter cassette comprising: a base editor region comprising at least one base editor target site; a second in-frame non-functional fluorescent reporter comprising at least one self-tolerizing peptide; an off-target array region comprising a known gene editing target site from at least one human gene; and a second out-of-frame functional fluorescent reporter. In accordance with these embodiments, the first and second reporter cassettes detect efficiency of a gene delivery system based on fluorescence of the at least one first or second out-of-frame functional fluorescent reporter.

### BRIEF DESCRIPTION OF THE DRAWINGS

FIGS. 1A-1B: H2B-GFP reporter pig. Representative photographs of tissues from H2B-GFP reporters (FIG. 1B). As demonstrated, the nuclear GFP is easy to score and validate in multiple tissues. It shows constitutive expression and high resolution, which facilitates quantitative image analysis due to the distinct nuclear staining compared to diffuse cytoplasmic staining. A schematic representation is provided in FIG. 1A, along with quantitative Western blot analysis.

FIGS. 2A-2E: Allogeneic engraftment of wild type pigs with H2B-GFP fetal liver cells. D42 wild type fetuses were injected with H2B-GFP hematopoietic stem cells and allowed to go to term. Thymuses were collected at 3 weeks post birth and analyzed for the presence of the donor (H2B-GFP) cells. As demonstrated, all pigs were engrafted. Also, the nuclear H2B-GFP cells can be easily distinguishable and quantitated using flow analysis.

FIGS. 3A-3D: Development of a pig model of Angelman Syndrome. (A) Schematic showing conserved UBE3A regions in pig (brown) and mouse (pink) relative to human. Schematic represents chain alignments. (B) Schematic of pig UBE3A gene and location of CRISPR targets (red boxes) in exons 1 (CRISPR-1) and 13 (CRISPR-2). (C) Schematic of UBE3A complex heterozygous mutation generated in porcine fetal fibroblasts. Mutations consist of 97 kb and 1 bp deletions of each allele of UBE3A. (D) Seven-month-old male boar with complex heterozygous UBE3A mutation (UBE3AΔ1bp/Δ97kb). Three boars are at Texas A&M University and being used to establish WT and AS animals for the proposed studies.

FIG. 4: Identification of OTE using CIRCLE-seq and GUIDEseq and comparison of SpCas9-WT and SpCas9-HF1. These are representative results obtained against two of the sites analyzed herein, VEGFA1 and FANCF2. (Figures are adopted from Tsai et al., 2017, and Tsai et al., 2014.)

FIG. 5: Representative examples of gene editing work with AAVs in the mouse brain. AAV2g9 carrying sgRNA to MIR137 were injected into Cas9 transgenic mice. Targeted cells (green) were identified in brain but not liver.

FIGS. 6A-6C: Results related to the validation of the reporter switch described herein. (A) Test of ability of reporter to detect differences between WT and HF1 SpCas9. Porcine fetal fibroblasts were transfected with a “traffic-light” type reporter expression vector containing a known high-frequency OFF-target site of FANCF2 combined with FANCF2 gRNA and either SpCas9-WT or SpCas9-HF1. Cleavage of the reporter at the known off-target site leads to expression of GFP. About 50,000 transfected cells were analyzed for fluorescence 48 hours after transfections. (B) Test for ACTB expression in non-dividing cells. ACTB expression levels are comparable in dividing and non-dividing cells. Actin expression detected by qPCR from log phase or serum starved (non-cycling) fetal fibroblasts (normalized to GAPDH). (C) Testing whole genome amplification kit. Whole genome amplification from 5 cells yields clean PCR products at 5 different genomic loci. Bands were sequenced, and all showed correct target.

FIGS. 7A-7I: Reporter constructs used to generate large animal models for evaluating gene editing technology. Detailed description for each functional component of the reporter constructs is provide herein.

FIGS. 8A-8C: (A) Exemplary data depicting comparison between ON-target cleavage and OFF-target cleavage for FANCF site 2 for wildtype SpCas9. On-target plasmids or off-target plasmids were co-transfected into porcine fetal fibroblasts with SpCas9 (Addgene #42230) and gRNA (Addgene #43861). Percentage was calculated by flow cytometry. (B) OFF-target reporter detects differences between WT and HF1 SpCas9. Porcine fetal fibroblasts were transfected with a “traffic-light” type reporter expression vector containing a known high-frequency off target site of FANCF2 combined with FANCF2 gRNA and either SpCas9-WT or SpCas9-HF1. Cleavage of the reporter at the known off-target site leads to expression of GFP. About 50,000 transfected cells were analyzed for fluorescence 48 hours after transfections. These results are highly similar to previously shown OFF-target frequencies from GUIDE-seq. Percentage was calculated by flow cytometry. (C) Percent of GFP positive porcine fetal fibroblasts when cells were co-transfected with the target indicator and either Cre or Base-Editor 3 expressing plasmids or the target indicator alone. Percentage was calculated by flow cytometry.

FIG. 9: Expression of NLS-mCherry when OFF-target indicator was co-transfected with CRISPR plasmid and gRNA targeting off-target site for FANCF2.

FIGS. 10A-10B: Exemplary indicator construct inserted into porcine genomic DNA. (A) PCR gels demonstrate genomic insertion of the construct into cellular DNA of porcine fetal fibroblasts. PCR primers are anchored both in the reporter insert (forward) and in the genomic DNA (reverse). This demonstrates the successful generation of a porcine genome with the indicator transgene integrated into the desired location in the ACTB locus. (B) Schematic plasmid map of an exemplary nucleic acid construct for the integration of an entire indicator construct into genomic DNA of an organism. The plasmid includes sequences for ON-target and OFF-target and associated features and relies upon regions of genomic homology for integration.

### DETAILED DESCRIPTION

Embodiments of the present disclosure provide compositions and methods related to the assessment of gene editing technologies in an animal model with single-cell resolution. In particular, the present disclosure provides a novel gene editing reporter system and transgenic animal platform for testing and optimizing gene editing technologies in vivo prior to implementation in humans.

Embodiments of the present disclosure include a gene editing reporter system for use in any model organism (e.g., pig) that will facilitate testing the on- and off-target rates of a range of gene editors (e.g., SpCas9, SaCas9, C2c1 and Cpf1, in addition to any DNA editors identified in the future) and the measuring of rates of a wide range of gene editing events (e.g., gene disruptions (non-homologous end joining-NHEJ), gene repair (homology directed repair-HDR), base editing, and gene insertions (homology independent targeted insertion—HITI)). Embodiments of the present disclosure also facilitate testing the efficiency and tissue/organ distribution of new targeted or non-targeted delivery systems whether they be viral or non-viral, as well as the testing to fetal and postnatal gene editing approaches. With gene editing enzymes being continuously being developed and improved, and novel delivery methods emerging, there is a growing need for cost-effective reporter systems capable of being adapted to large animal model systems, which can be used to develop safety data before human clinical trials. Availability of the various embodiments of the present disclosure will facilitate the rapid in vivo testing of new gene editing technologies and therapies.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, the term “animal” refers to any animal (e.g., a mammal), including, but not limited to, humans, non-human primates, pigs, rodents (e.g., mice, rats, etc.), flies, and the like.

As used herein, the term “non-human animals” refers to all non-human animals including, but are not limited to, vertebrates such as rodents, non-human primates, ovines, bovines, ruminants, lagomorphs, porcines, caprines, equines, canines, felines, ayes, etc.

The term “transgene” as used herein refers to a foreign, heterologous, or autologous gene and/or fragment thereof that is placed into an organism (e.g., by introducing the gene into newly fertilized eggs or early embryos). The term “foreign gene” refers to any nucleic acid (e.g., gene sequence) that is introduced into the genome of an animal by experimental manipulations and may include gene sequences found in that animal so long as the introduced gene does not reside in the same location as does the naturally-occurring gene.

As used herein, the term “transgenic animal” refers to any animal containing a transgene.

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full-length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example, a 24-residue oligonucleotide is referred to as a “24-mer.” Oligonucleotides can form secondary and tertiary structures by self-hybridizing or by hybridizing to other polynucleotides. Such structures can include, but are not limited to, duplexes, hairpins, cruciforms, bends, and triplexes.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “peptide” refers an oligomer to short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein or a non-natural (artificial) sequence.

As used herein, the term “polypeptide” refers to a polymer of amino acids linked together by peptide bonds that is greater than about 50 amino acids in length. Polypeptides may comprise natural amino acids, non-natural amino acids, amino acid analogs and/or modified amino acids, and may be a naturally occurring sequence, or a non-natural (artificial) sequence, or a subsequence of naturally occurring protein or a non-natural (artificial) sequence.

“Sequence identity” refers to the degree two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families, e.g., acidic (e.g., aspartate, glutamate), basic (e.g., lysine, arginine, histidine), non-polar (e.g., alanine, valine, leucine, isoleucine, proline, phenylalanine, methionine, tryptophan) and uncharged polar (e.g., glycine, asparagine, glutamine, cysteine, serine, threonine, tyrosine). The “percent sequence identity” (or “percent sequence similarity”) is calculated by: (1) comparing two optimally aligned sequences over a window of comparison (e.g., the length of the longer sequence, the length of the shorter sequence, a specified window), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Reporter Constructs and Methods of Use

Embodiments of the present disclosure include the generation of reporter constructs and animal models to evaluate the safety and efficacy of gene editing technology. In some embodiments, cells can be generated, reporters can be extensively tested, and off-target effects (OTE) in the genome of an animal model (e.g., pig genome) can be identified. Data generated using these systems and methods can help evaluate and validate a reporter system in vivo. In some embodiments, results described herein include gene editing in pigs, use of CIRCLE-seq, use of AAVs in vitro and in vivo, and whole genome amplification. In some cases, both male and female cell lines, fetuses and pigs can be examined to determine any potential sex effects (sex as a biological variable).

Human targets. The positives of using human targets include, but are not limited to, being able to compare to existing published information, providing more accurate information as to how a particular gene editing approach will perform in humans, and this facilitates application of the information to the clinic. Adding additional target sites is not a significant obstacle, as the costs of generating the animal per se are not affected. Advantages include having multiple targets to choose from, each of which often has published information associated with it, which is useful for making comparisons.

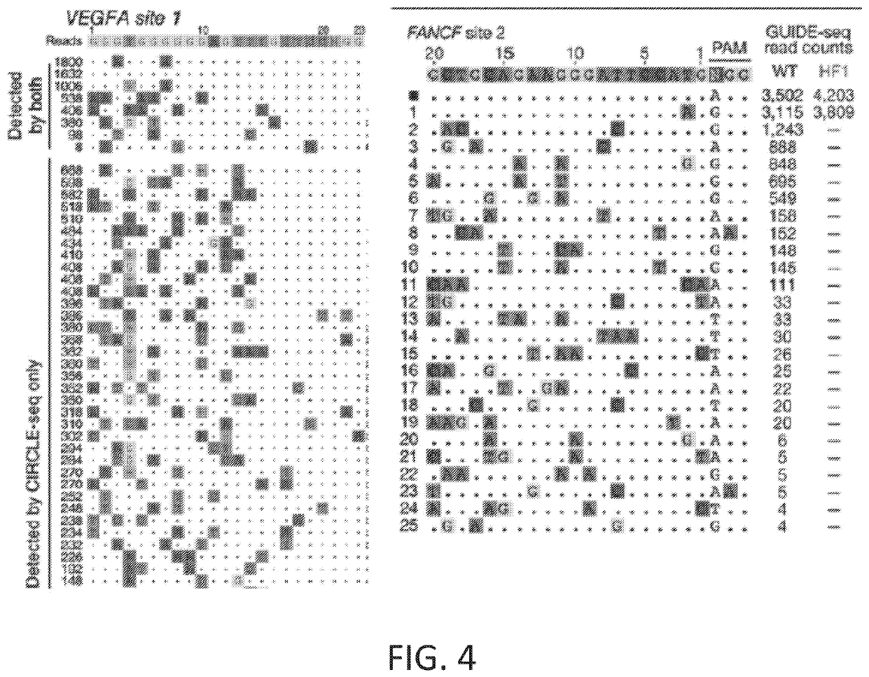

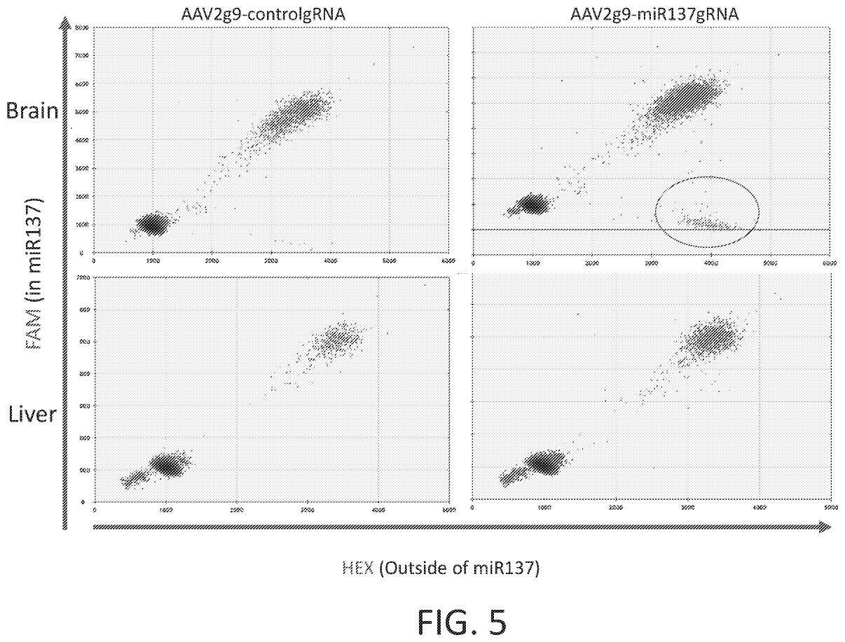

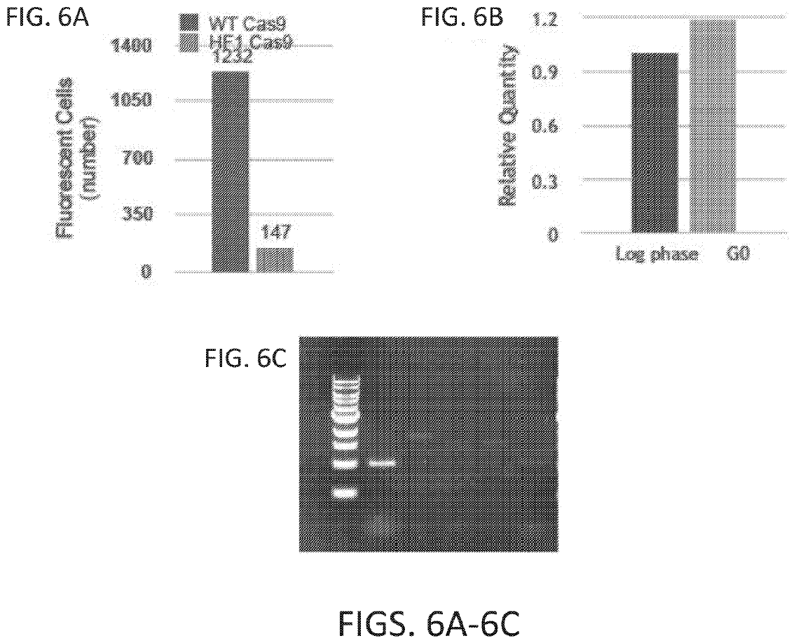

Use of SpCas9-WT and SpCas9-HF1. As has been shown in preliminary results (FIG. 4), OTE to the FANC2 targets have been described for both enzyme types. This provides a unique opportunity to both test the OFF-target reporter in vivo and in vitro, as well as allows for comparisons between the in vitro generated data with in vivo data.

Use of SCNT-generated D40 fetal fibroblasts. Being able to use SCNT to generate D40 pregnancies for completion of the in vitro experiments significantly improves the timelines required for completion of a project without impacting the quality of the data. While it is known that SCNT introduces epigenetic artifacts into the genome, even if present, those artifacts will not affect the eventual outcome of gene editing. Using two highly divergent chromatin architectures, the active versus the silent imprinted gene loci, it has been shown that while the kinetics of gene editing may be impacted by chromatin heterogeneity, the eventual outcome is not. Under the Cas9 concentrations normally seen by nucleofection or AAVs expression in vitro, all gene edits should be completed by 48 hr regardless of the chromatic conformation. As described further herein, collecting 5-14 days post-transfection ensures complete targeting. Moreover, critical in vivo experiments, where Cas9 concentration may indeed be an issue, can be carried out using naturally bred animals.

Use of ACTB instead of ROSA26 as a safe harbor. A commonly used ubiquitously expressed safe harbor, is the ROSA26 locus. This locus has been widely used in mice and more recently in swine. However, there have been reports that ROSA26 expression can vary widely in particular in certain cell types. For example, previous reports indicated that expression levels of lacZ from the ROSA26-lacZ reporter mouse changed drastically during remodeling of arteries, with variability in beta-galactosidase positivity among ROSA-LacZ organs. Of greater concern for transplantation or cell tracking studies is the discrepancy between ROSA26 locus expression and actual cell tracking. For example, Theise and colleagues (Theise, 2003) reported that after bone marrow transplantation of ROSA26-lacZ cells into irradiated mice, splenic engraftment was 90% when measured by Y-chromosome analysis but only 50% when measured by lacZ staining. This suggests that under certain conditions the ROSA26 promoter will give inaccurate information of the degree of in vivo gene editing. Similarly, when using the ROSA locus as a safe harbor it has been reported that this locus is prone to promoter interference and orientation dependence. An additional concern of the ROSA26 is the potential to affect expression of deleterious genes located near the ROSA26 locus. In particular, oncogenes such as SRGAP3 are located near the ROSA26 locus in humans, mice and this genomic organization is conserved in pigs.

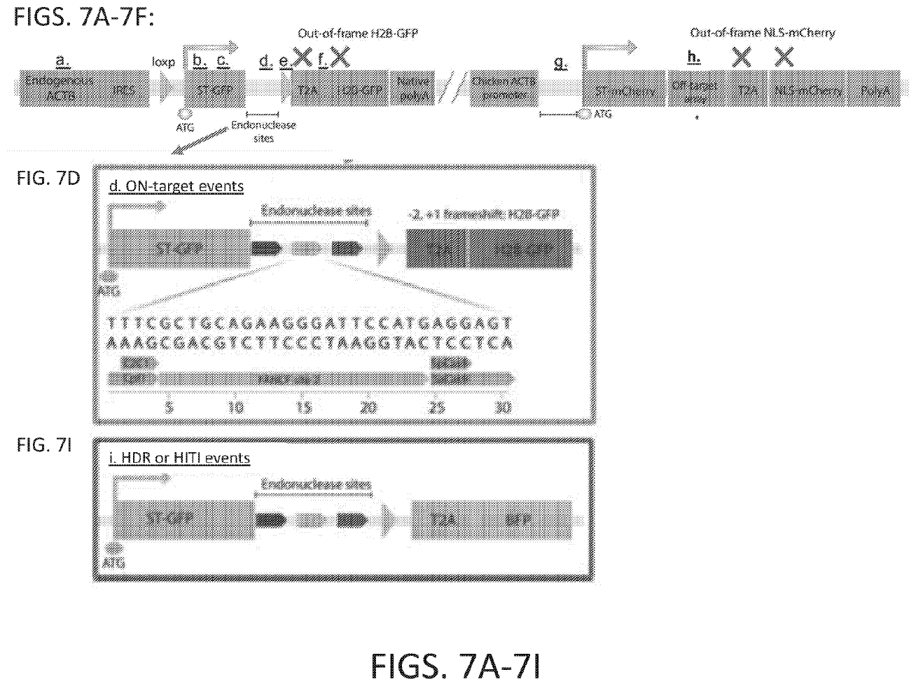

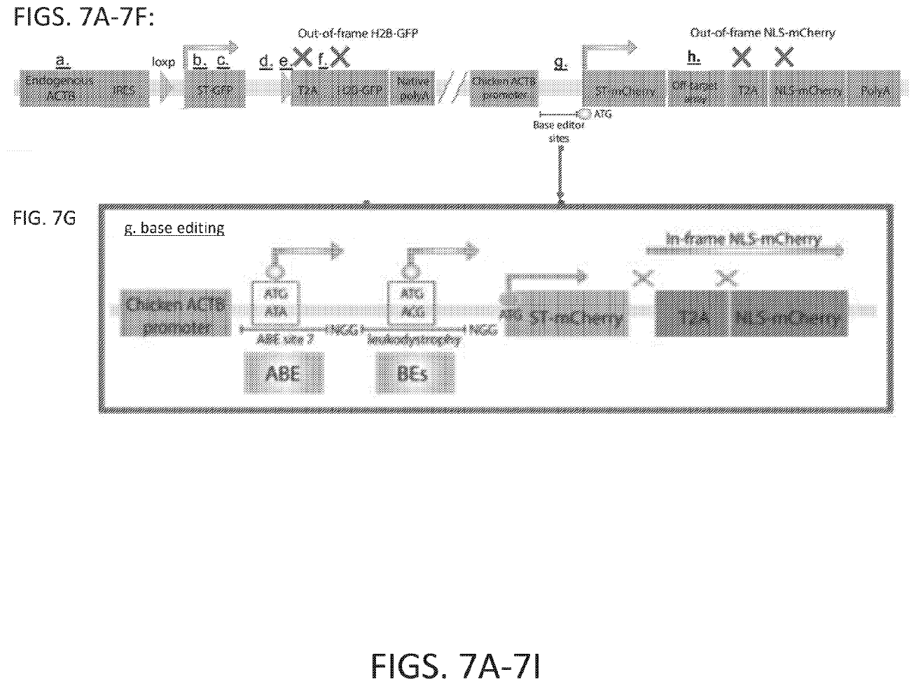

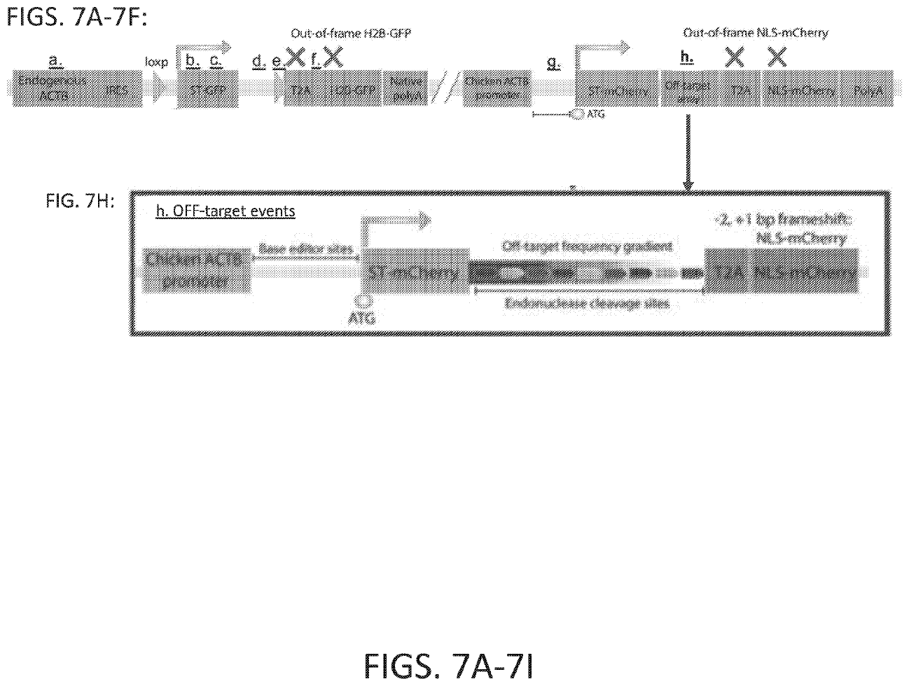

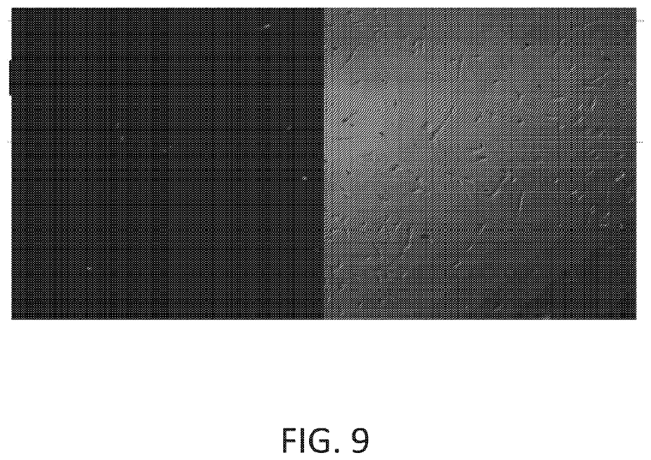

Placing both ON- and OFF-reporter in a single ACTB allele versus in separate ACTB alleles. The benefits of placing both reporters in tandem, as described herein, is that all events can be easily scored at single cell resolution (FIG. 7 and FIG. 9). In addition, from a practical long-term perspective, breeding of the establish lines and generation of test animals is much simpler as they only have to inherit one allele. Thus, by breeding a homozygous reporter boar to a wild-type sow, 100% of the offspring will contain exactly one copy of the reporter, and therefore will all be usable test animals. Placing the ON- and OFF-reporter in separate alleles reduced this number to 25%. This is not a trivial difference with existing FDA guidelines that require all offspring from gene edited animals, whether gene edited themselves, be treated and disposed of as gene edited. This can complicate logistics as well as increase operating cost to both the production and the testing center. One drawback is that by placing both reporters in the same allele, it is more difficult to measure genomic translocations. It is also acknowledged that placing the reporters in tandem can introduce the possibility for a deletion of the intervening region if both ON- and OFF-target sites are cleaved. However, the OFF-target reporter (mCherry) will still be switched on and detectable by sequencing.

Reporter Design. As shown in FIG. 7, the reporters were designed based on the well-known and characterized (Certo et al., 2011) “traffic light” reporter that has been used to examine gene editing in a variety of systems. In one embodiment described herein, the reporter (H2B-GFP or nls-mCherry) is off-frame by and is only active if indels place it in frame. This allows observation of 1 out of 3 possible events, which are taken into account in the calculations. Both the ON- and OFF-target reporters can identify NHEJ indels, HDRs, HITI, and base editing at single cell resolution. As depicted in FIG. 7, ON-target events can generate green nuclei, while OFF-target events can generate red nuclei. ON- and OFF-target events in the same cell without deletion of an intervening region can result in yellow nuclei, while ON- and OFF-target events in the same cell with deletion of intervening region can result in deletion of H2B-GFP and expression of mCherry only (red nuclei). Thus, both OTE with or without a deletion induce expression of the mCherry marker. HDR or HITI will result in mTAGBFP2. The three markers were selected in part because they do not have spectral overlaps and can be easily separated by flow.

Features of the reporter constructs provided herein include, but are not limited to, the following:

(a) Safe harbor. Use of the ACTB locus as a safe harbor as well as to drive the ON-target reporter. Using the endogenous ACTB promoter to drive a reporter results in ubiquitous expression of the H2B-GFP locus without any deleterious health effects in pigs has been demonstrated. The ON-reporter can be driven by the endogenous ATCB promoter using the same target sequence used to generate the H2B-GFP reporter pig. The OFF-target reporter can be downstream of the endogenous ACTB but within the region identified as a safe harbor. It can be driven by the chicken ACTB promoter or any other constitutive promoter. We chose this promoter, which was chosen in part to ensure all the proper regulatory elements that drive ACTB are within the locus. In some cases, enhancer elements such as the WPRE were excluded in order to keep expression levels low

(b) Self-tolerizing peptides for reporter proteins. An in-frame GFP (ON-reporter) or mCherry (OFF-reporter) peptide combination that do not express a functional protein but are known epitopes and will tolerize the pig to GFP/mTAGBP2 or mCherry (Brusic et al., 2004). This can be important, as others have shown that expression of markers such as GFP in a naïve animal can result in immune rejection of the GFP-expressing cells. Investigators using reporter mice that activate GFP or luciferase in an immune competent mouse have demonstrated that this can drastically affect interpretation of the results and have incorporated antigenic epitopes that can tolerize the mice to both reporters (Ju et al., 2015). Embodiments of the systems provided herein incorporate these features, as there is a need to carefully analyze the effects of the immunogenicity of the delivery system and/or the expression of, e.g., CRISPR variants. Any reporter system that, when activated, can itself induce even a minor immune response would be unreliable and negatively impact the rigor and reproducibility of the data generated.

(c) Novel editors (“landing pads”). Unselected “random” DNA targets that can be used to test new editors that have rules of targeting that differ from those of SpCas9, SaCas9, Cpf1, or C2C1. The sequence encoding the self-tolerizing peptides contains a variety of potential PAM sequences on either forward or reverse strands that can act as the targets for new editors. For example, an animal can still be tolerized to GFP and mCherry peptides during lymphocyte development regardless of somatic targeting or disruption of these sites. As these are unknown new targets, they will only work to detect ON-target effects as it is impossible to predict what the new rules of binding will be. To examine OFF-target effects of these new editors, CIRCLE-seq could be used, for example.

(d) Well characterized human target sites. In the embodiments described herein, three CRISPR-Cas9 targets sites were selected for further experimentation and evaluation: the human FANCF2, VEGFA1, and HEK1 targets. These three sites have been extensively studied, and ON-and OFF-target frequencies evaluated in vitro using both GUIDE-seq and CIRCLE-seq. In addition, FANCF2 has been used to compare SpCas9-WT with SpCas9-HF1. This is important for validating both the ON- and OFF-target reporters of the present disclosure. Finally, none of the sites selected match any known region of the porcine genome supporting that the effects will be limited to the reporter region and will not affect the pig FANCF, VEGFA or the HEK293 intergenic region (HEK1). This adds rigor to the systems and methods described herein, as it allows comparison of the results with those of other laboratories, thus ensuring that the data generated is of high quality and reproducible. However, it is not unexpected that the editor may generate previously unidentified OTE in the pig genome, which can be identified using CIRCLE-seq.

(e) Cre-inducible testing site. In one embodiment, the ON-target reporter can include loxP sites flanking the GFP/mTAGBFP2 tolerizing peptides. These have been placed such that excision places the H2B-GFP in frame. This can be more effective than the CRISPR-Cas for testing the system as it identifies all Cre-excision events while CRISPR-Cas editing (indels) identify only 1 out of 3. Additionally, AAVs containing Cre are available allowing in vivo testing of the reporter independent of gene editing. This allows rapid examination of the biodistribution of AAVs or any new delivery methods.

T2A peptide linked to the H2B-GFP or mCherry. As one of ordinary skill in the art would recognize based on the present disclosure, embodiments include a traffic light reporter capable of nuclear localization. The nuclear localization can be added to enhance reproducibility and sensitivity, as cytoplasmic fluorescent markers can be difficult to score accurately due to high auto fluorescent background in certain tissues. Data supports the use of this reporter and the ease of scoring and interpretation of both IHC and flow data. This again adds rigor and reproducibility to the present systems. Additionally, 2A self-cleaving peptides, or 2A peptides (e.g., T2A peptides), include 18-22 amino acid-long peptides, which can induce the cleaving of a recombinant protein in a cell. In some cases, 2A peptides are derived from the 2A region in the genome of virus; however, as evident throughout the present disclosure, any self-cleaving peptides can be used.

(g) Base editor on-switch. This allows the use of a distal ATG site as the main site for the off-frame “traffic-light” reporter and the creation of a proximal base editing reporter site that upon base editing can create a new proximal ATG that will place the traffic light reporter in frame and result in expression of the NLS mCherry or H2B-GFP (both nuclear).

(h) Identifying HDRs with small oligos versus NHEJ indels. When using small oligos (<100 bp) for HDR gene editing, a reporter can be placed in frame and express H2B-GFP. Phenotypically, it is difficult to differentiate from NHEJ induced indels that will also turn H2B-GFP. To separate small oligo HDR from NHEJ events, a target region can be sequenced after WGA.

(i) Evaluate frequency of HDR/HITI vs NHEJ. Successful insertion of mTAGBFP2 in place of H2B-GFP can lead to blue fluorescence, whereas unsuccessful knock-in events can lead to NHEJ indels at the target locus and therefore H2B-GFP expression. Frequency of each event can be compared by evaluating the fluorescence of each reporter.

(j) OFF-target frequency gradient. The OFF-target sequence array is composed of known OFF-targets of FANCF2, VEGFA1 and HEK1. The frequency of gene editing at the selected sites has been well studied and compared when using wild type or high-fidelity Cas (FIG. 4). This allows a direct comparison of the in vivo data with existing in vitro literature. Moreover, an array in an OTE frequency gradient has been generated (FIG. 7) so it has one high frequency OTE (25% of ON-target frequency), one mid-frequency OTE (5-10% of ON-target frequency) and one low frequency (1-5% of ON-target frequency) for each of the FANCF2, VEGFA1 and HEK1 sites. The targets are arranged in tandem by frequency (high-mid-low) with a 90 bp spacing between each triplet (FIG. 7). This allows identification of indels less <40 bp which covers the majority of known CRISPR-Cas9 induced NHEJ indels. This essentially results in an OT reporter with a sensitivity gradient.

Embodiments of the present disclosure also include the use of open-source CIRCLE-seq package (Tsai et al., 2017) to process the sample-specific paired end FASTQ files and to produce the list of CIRCLE-seq detected off-target cleavage sites and the corresponding read quantification. Stem-leaf plots, bar charts, and boxplots will be generated to display the distributions of CIRCLE-seq read counts and relative frequencies for on-target and off-target sites. Fisher's exact test and the Wilcoxon rank-sum test can be used to compare the frequencies of on-target and individual off-target sites between different conditions. Correlations between the overall frequencies of on-target and off-target sites across different conditions can be assessed using Spearman rank correlation.

Generation and in vitro validation of gene editing reporter and identification of OTE in the pig genome. To ensure that the reporter system works as intended, and to generate critical comparative baseline data with respect to OTE in the pig genome, cell lines can be generated and in vitro characterized can include the following:

Fetal fibroblasts (FF) cell lines carrying ON- and OFF-target reporters in the ACTB locus. Both male and female Yucatan lines and mono- and bi-allelic HDR-mediated knock-ins can be generated. Gene editing can be carried out as described. Mono-allelically targeted cells can be used for generation of D40 fetal fibroblast by SCNT. In addition, if identifying bi-allelically modified cells after screening of 100 colonies is difficult, mono-allelically modified fibroblasts can be used for a new round of knock-ins to generate the bi-allelic reporter cell lines required to produce founder animals.

Comparison of frequencies of ON- and OFF-target effects. Using SpCas9-WT or SpCas9-HF1, ABE and BE3 base editors, cells can be flow sorted on the basis of reporter expression and compared to determine the relative frequencies of OTEs. Comparisons of ON- and OFF-targeting frequencies can also be used to validate ON- and OFF-target reporters. For example, SpCas9-Wt and SpCas9-HF1 (n=2), gene editing via nucleofection or AAVs (n=2), and three targets (FANCF2, VEGFA1, and HEK1: n=3) can all be compared in two independent FF lines—one male and one female— (n=2; Total n=24).

Methods. Gene editing by nucleofection can be done as described in Tsai et al., (2017). Gene editing via AAV viruses can be as described herein; in some cases, cells can be kept for 2 weeks prior to collection of cells for analysis. Calls can be infected at a MOI of 10E4 GC/cell. For 5E5 cells, 1E9 AAV GC can be used.

For experiments involving both nucleofection and AAVs, a total of 500,000 cells/test can be used, and at least 100,000 cells can be analyzed by flow. Frequencies of GFP+, mCherry+, H2B-GFP+/mCherry+ and double negatives (GFP−/mCherry−) can be calculated. In addition, single cells (10/category) can be manually picked and used for whole genome amplification as described herein. After amplification, the reporter region as well as selected OTEs identified can be amplified and sequenced. This will allow identification and quantification of the type of gene edits in each population (single positive, double positive and double negative) at a single cell level.

Base editing validation. For base editing, two target sites are included to accommodate base editors eliciting either a substitution of C-G to T-A (BE3) or a T-A to C-G (ABE). For BE3, a target site was selected from the human genome associated with Hypomyelinating Leukodystrophy 2, harboring a SNP (T→C) located in the editing window (Komor, 2016). Successful editing events convert the C into a T, thereby creating a new ATG in frame with downstream nls-mCherry. For ABE, the previously established “ABE site 7” was chosen (Gaudelli, 2017), which contains an A in the editing window. Successful editing events convert the A into G, creating a new in-frame ATG for nls-mCherry expression. OTE in the pig genome of these two targets can also be analyzed using the same sgRNA but with wild type Cas9 and the cells can be analyzed by CIRCLE-seq as described above. This provides additional regions to examine base editing OTE (after in silico selection of those that are amenable to base editing). Overall, two independent lines will be used (one male, one female; n=2), two delivery methods (n=2) and two base editors (2) to examine base editing efficiency (Total n=8). In addition, to determine OTE of the two-base editing sgRNA, 2 sgRNAs, 1 cell lines, 1 delivery method (AAVs) and the wild type Cas9 (Total n=6) will be examined.

To validate the ability to detect NHEJ repair versus HDR/HITI. Clinically, correction of short regions of DNA containing disease-inducing mutations using short oligos and HDR will be one of the main uses of this technology. In some cases, when using HDR and short oligos, it is difficult to discriminate visually between HDR and NHEJ insertion as both will turn on the traffic-light reporter. However, by comparing the frequency with and without the HDR oligo, as well as by sequencing single cell events to identify the target region indel sequence, it is possible to determine the approximate frequencies of HDR events. For these experiments, the FANCF2 site can be used. Additionally, in one embodiment, 500,000 cells can be edited as described above with the exception that a 100 bp oligo with homology to the target region, but designed to place the traffic light reporter in frame will be added to the nucleofection transfection mix or the AAVs.

For large HDR/HITI mediated knock-ins, the OFF-frame H2B-GFP will be replaced with an on-frame T2A-nls-mTAGBFP2, or it will be inserted (HITI). This will discriminate between HDR/HITI (blue FP) and NHEJ (GFP). mTAGBFP2, a blue fluorescent protein, is spectrally distinguishable from GFP and mCherry. As GFP and mTAGBFP2 are 95% similar at the protein level, the self-tolerizing GFP sequence will also tolerize to mTAGBFP2. Additionally, 500,000 cells can be edited as described above with the exception that a homologous recombination template or HITI template containing the mTAGBFP2 and homology to the target region can be included, which will be added to the nucleofection transfection mix or the AAVs.

For both nucleofection or AAVs, at least 100,000 cells can be analyzed by flow. Frequencies of GFP+, mCherry+, and mTAGBFP2+ can be calculated. In addition, single cells (20/category) can be manually picked and used for whole genome amplification and analysis. Overall, two independent lines (one male, one female; n=2), two delivery methods (n=2), one endonuclease-SpCas9-HF1 (1), HDR or HITI (2) will be used (Total n=8).

Identification of FANCF2, VEGFA1 and HEK1 OTE in the pig genome. Using cells generated as described herein, SpCas9-WT and SpCas9-HF1 genomic OFF-targets events to the three selected targets can be identified using CIRCLE-seq.

Generation and in vivo validation of gene editing reporter pigs. The key questions that need to be addressed regarding in vivo gene editing center around the efficiency of the method being tested (both frequency and tissue tropism), its fidelity (OTE) and its safety (biological consequences). Three in vivo methods can be used (1 fetal and 2 postnatal) to fully validate the reporter. Combined, the three systems will provide a comprehensive set of data that can be used by others to select the testing method that best meet their needs. Each method has its own strengths and weaknesses but by generating a detailed comparison of the results from each, the information can be used to assess which testing method(s) better addresses the questions being asked. Rationale for using each of the three proposed methods are provided below.

Fetal injection (FD40). For the purposes of several of the embodiments described herein, injection into the pig fetus at FD40 of gestation provides multiple advantages, including, but not limited to the following:

(a) Reduced cost and increased efficiency. Due to the isolated nature of the uterus, the size of the fetus at this stage and the ability to inject multiple fetuses per pregnancy, a single pregnancy can be used to obtain multiple biological replicates per test gene editor or delivery system. This will result in decreased costs, increased ease of management of multiple projects, and reduction in the time required for validation or testing.

(b) Ability to target different tissue compartments. When using AAVs in fetal mice, amniotic fluid injection targets skin and digestive system, injection of leg targets muscle compartment, and liver injection targets liver and hematopoietic system. In addition, direct injection into the brain is also possible. Widespread AAVs transfection in the brain of NHP by fetal injection is also possible.

(c) Due the rapid growth of the fetus, gene editing at FD40 will provide a high degree of sensitivity compared to injection post birth and is likely to be a better predictor of long-term effects.

(d) By injecting at FD40, a period prior to the development of the immune system, it allows for induction of tolerance to any component of the delivery system (e.g., AAVs) or the reporter proteins being expressed. This will facilitate separation of immune effects from other effects when the data is compared to postnatal injection.

(e) A fetal injection model provides invaluable data related to ON- and OFF-target effects related to fetal treatments of genetic disorders in humans.

(f) By being able to use SCNT pregnancies from cell lines generated, as described herein, fetal in vivo testing can be initiated prior to establishment and breeding of the reporter lines.

D4 postnatal Injection (PD4). AAV inoculation at D4 was chosen, as this provides the piglets time to stabilize after cesarean and transfer to available pig bio-isolators. This approach includes, but is not limited to, the following advantages:

(a) Provides an immune competent host (as opposed to pre-immune fetal injections).

(b) Provides reduced housing costs. By using this age group, 4 piglets per bio-isolator can be housed for up to 4 weeks in a contained, safe, environment. From a testing perspective, it also adds rigor and repeatability by removing the maternal effect and allowing the animals to be raised in a highly controlled sterile environment. This will greatly facilitate comparison of data over time.

(c) Provides practical in vivo testing before scale-up. With delivery systems such as AAVs, it is possible to infect 1 kg piglet with 1E13 GC. Injection of a 50 kg pig would require 5E14 GC and cost approximately $20,000/pig in reagents alone. While large pigs may be one of the eventual targets, testing in the newborn pig can facilitate screening of gene editing methods in reporter animals before this expensive scale-up is undertaken.

D30 postnatal Injection (PD30). PD30 was chosen, as this provides a weaned pig that is approximately 10 kg. Advantages include, but are not limited to, the following:

(a) Fully developed immune system.

(b) A reasonable cost, scale-up in vivo testing system.

Generate founder pigs via Somatic Cell Nuclear Transfer (SCNT). Using cell lines generated and validated, offspring will be generated for completion of various experiments. One pregnancy will be generated that is expected to produce 4-6 offspring. Offspring can be maintained until breeding age for semen collection. Semen can then be shipped to the testing center for establishment of the testing lines. In addition, generated boars can be used to produce fetuses and animals as described herein.

Comparison of in vivo FANCF2 ON- and OFF-target effects of SpCas9-WT and SpCas9-HF1 after amniotic fluid and brain injection of AAV into FD40 fetuses. Fetal injections can be used, as described previously. Gene editing at FD40 can be done using ultrasound assisted methods. On average, 4-6 fetuses per pregnancy can be injected, and pregnancy losses after injection are generally less than 5% (n>100 injections). Fetuses can be injected at FD40 and collected 3 weeks post-injection. Multiple tissues can be analyzed by IHC as well as after single cell isolation and flow separation. ON- and OFF-target frequencies can be compared to those obtained in vitro.

Utilizing heterozygous reporter cell lines and SCNT to generate the fetuses is also feasible. Experimental design can include the following: For measuring NHEJ, two editing SpCas9 (wild type and HF1), 2 injection sites (amniotic fluid and brain), 3 fetuses and one-time point (3 weeks post injection) can be examined. For measuring HDR, one editing SpCas9 (HF1), 2 injection sites (amniotic fluid and brain), 3 fetuses and one-time point (3 weeks post injection) will be examined. For measuring HITI, one editing SpCas9 (HF1), 2 injection sites (amniotic fluid and brain), 3 fetuses and one-time point (3 weeks post injection) will be examined. For measuring base editing, two base editors (BE3 and ABE), 2 injection sites (amniotic fluid and brain), 3 fetuses and one-time point (3 weeks post injection) will be examined.

Methods for in vivo investigations. The methodology chosen to validate the reporter animals is designed to address the frequency and type of in vivo gene editing events including OTEs, to identify regional or cell type specificities of delivery methods, and to identify any inflammatory/immunological responses to gene edited cells. In some embodiments, the methodology includes the following:

(a) AAVs dosage will be 1E12 for amniotic fluid injection, 1E11 for direct brain injection, 1E13 and 1E12 for systemic and brain injection, respectively, into 1 kg pigs, and 1E14 and 1E13 for systemic and brain injection into 10 kg pigs. Dosages have been calculated on the basis of previous postnatal and fetal injection experiments in pigs and NHP primates.

(b) Frequency and type of editing will be carried out as described herein. For example, tissues collected from liver, lung, kidney, and brain from fetuses or postnatal pigs can be single-cell dissociated, populations separated based on spectral fluorescence as described previously, and frequencies calculated after examining at least 100,000 cells. For identification of type of gene edits and examining OTEs, 10 cells/category will be WGA and the same regions that were analyzed previously will be examined and sequenced.

(c) Regional and cell type distribution of gene edits. Histological analysis can be carried out in the same tissues (liver, spleen, kidney and brain) on frozen sections. Fluorescence from the H2B-GFP pigs is maintained with high fidelity in frozen sections. This allows for the examination of how gene edits are distributed within each tissue type and whether certain cell types are preferentially edited.

(d) Immune responses. Frozen sections can be analyzed above for the presence of signs of inflammatory responses including neutrophil and/or macrophage infiltration.

Comparison of in vivo FANCF2 ON- and OFF-target effects of WT SpCas9 and SpCas9-HF1 after brain or systemic injection of AAV into postnatal D4 (PD4) pigs. For measuring NHEJ, two editing SpCas9 (wild type and HF1), 2 injection sites (systemic injection and brain), 2 piglets (one male, one female) and one-time point (3 weeks post injection) will be examined. For measuring HDR, one editing SpCas9 (HF1), 2 injection sites (systemic injection and brain), 2 piglets (one male, one female) and one-time point (3 weeks post injection) will be examined. For measuring HITI, one editing SpCas9 (HF1), 2 injection sites (systemic injection and brain), 2 piglets (one male, one female) and one-time point (3 weeks post injection) will be examined. For measuring base editing, two base editors (ABE and BE3), 2 injection sites (systemic injection and brain), 2 piglets (one male, one female) and one-time point (3 weeks post injection) will be examined.

Comparison of PD4 vs. PD30 responses to FANCF2 gene editing with SpCas-WT. Due to the high costs of delivering AAVs to large animals, in some embodiments, validation of the PD30 model can be limited by testing SpCas9-WT (n=1), NHEJ and HDR or HITI, depending on results of the experiments described herein (n=2), two pigs (n=2) and one site, systemic (n=1) (Total n=4). Weight of a Yucatan at postnatal PD30 is approximately 10 kg.

In accordance with the embodiments described herein, compositions, systems, and constructs of the present disclosure can be used for various applications, including but not limited to the following: Detection of on and off target gene editing events in cells (e.g., using plasmid-based reporter), including measuring the rate of correct and/or incorrect editing at single cell resolution. Detection of base-editing gene editing events in cells (e.g., using plasmid-based reporter). Comparison of high-fidelity or wild-type off-target efficiencies (e.g., using plasmid-based reporter). Detection of Cre-mediated recombination (e.g., using plasmid-based reporter). Comparison of integration efficiency or homologous recombination efficiency vs. NHEJ (e.g., plasmid or genomic). This can include comparing the functionality of existing and also newly developed gene delivery methods, and/or exiting or novel gene editing systems. Detection of the frequency of gene editing events, at a single cell resolution in every cell of an organism (fetal or adult), with gene editors being delivered to cells or tissues ex vivo (e.g., take cells from various tissue types, culture them, and then transfect them with the editors to evaluate editing effects). This includes measuring the tissue/cell distribution of gene edits (both on and off targets) in a live organism. Determining clonal expansion of specific cells by sequencing indels generated by the gene editor in the reporter On and Off target sites as well as selected genomic OT sites. Performing lineage tracing analysis by examining the segregation patterns of indels in the On, Off reporter sites and genomic OT sites. Detection the frequency of gene editing events, at a single cell resolution in every cell of an organism (fetal or adult), with gene editors being delivered to cells or tissues in vivo. This application has particular commercial relevance, as it would allow for development and testing of existing and novel in vivo delivery methods for clinical applications in humans, such as: existing or novel viral delivery systems; existing or novel non-viral delivery systems; existing or novel tissue trophic delivery systems; and systemic versus local delivery.

### 3. Examples

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

Generation of Detailed Characterization of Gene Edited Pigs Expressing Nuclear GFP in All Cells in the Body. The development of fluorescence proteins as molecular tags has allowed complex biochemical processes to be correlated with protein functionality in living cells. In addition, genetic engineering of encoded biological fluorescent proteins has marked an evolution in the field of stem cell biology, allowing the development of cell-traceable systems and the ability to track the fate of adult stem cells for therapeutic purpose in biomedical models. Among these molecular tags, the most widely used one is the green fluorescent protein (GFP) from the jellyfish Aequorea victoria. Based on this concept, transgenic mice, rats, rabbits and pigs expressing eGFP under a variety of conditions have demonstrated their usefulness in basic and translational research. Transgenic pigs harboring and expressing green fluorescent proteins under different conditions have been described. However, identification and quantification of engrafted donor cells after cell/tissue transplantation remains challenging due to strong auto-fluorescence, especially when GFP is expressed in the cytoplasm. In addition, the diversity of cell phenotype and shapes make it difficult to distinguish/count GFP-positive donor cells when utilizing automated systems. This difficulty can be overcome via nuclear GFP labeling, allowing easy and convenient cell tracking after stem cells/tissue transplantation studies. Nuclear localization of GFP can be achieved by addition of a nuclear localization signal peptide or by fusion of GFP with proteins of the nucleosome core such as histones (e.g., H2B).

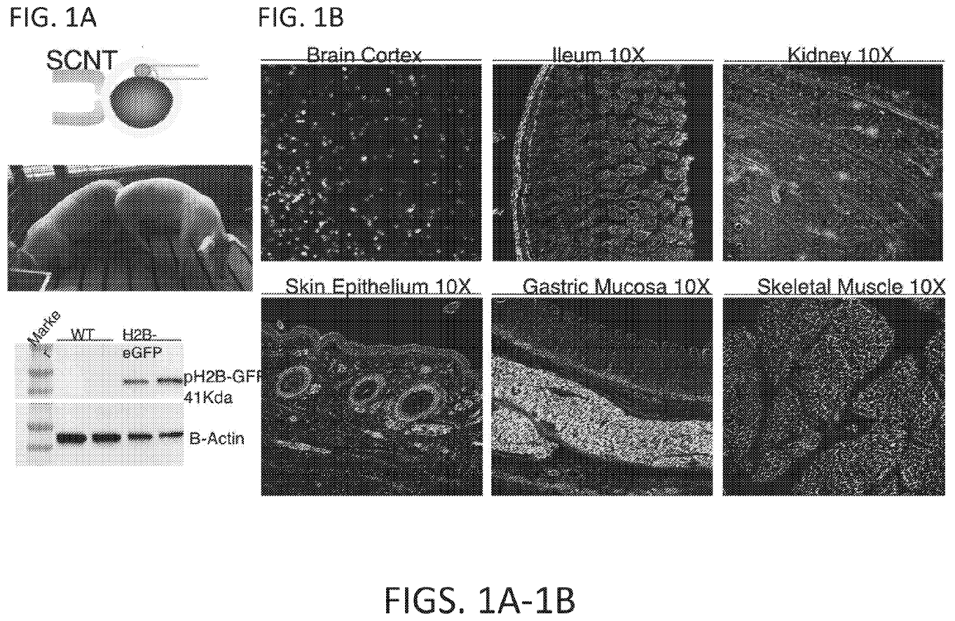

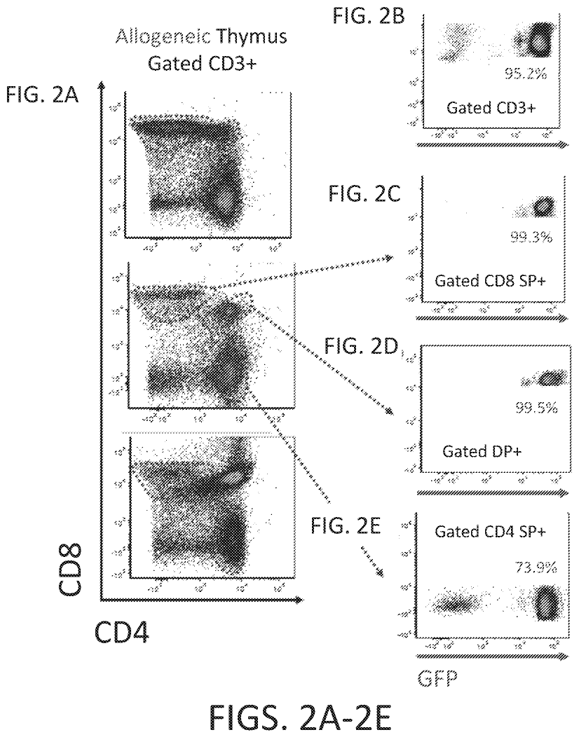

H2B-GFP expression in cell lines or transgenic mouse models have been described and shown to be of great value in the field of stem cell tracking, cancer biology and chromosome dynamic studies. On the basis of this information, the present disclosure includes the only existing H2B-GFP reporter pig to assist in ongoing studies on allogeneic and xenogeneic transplantation. The relevant preliminary evidence provided herein includes the use of CRISPR-Cas9 mediated gene editing to introduce a reporter into a specific site in the pig genome and generate live pigs after SCNT (FIG. 1); the use of the Actin B (ACTB) locus as both an excellent ubiquitous promoter and a safe harbor. The use of ACTB as a safe harbor had been reported in mice but not yet in pigs (this site has considerable advantages over the commonly used ROSA26 locus); and extensive characterization of the model showed that H2B-GFP can be detected in all tissues tested, is easy to score, and can be detected in frozen sections, fixed tissues and by flow cytometry (FIG. 2).

### Example 2

Generation of Severe Combined Immunodeficient Pigs: Allogeneic Transplantation of H2B-GFP Hematopoietic Stem Cells (HSCs) into FD40 IL2RG/RAG2 DKO Fetuses. Pigs lacking IL2RG and RAG1 have been generated and have used for both allogeneic and xenogeneic hematopoietic transplantation studies. This line includes two sequential mutations starting with IL2RG followed by RAG2. To mutate the X-linked IL2RG locus, porcine fetal fibroblast (PPF) cell lines were co-transfected with TALENs targeting the junction between the signal peptide and the extracellular region. Analysis of single cell-derived colonies identified IL2RG mutants at an 8.5% frequency. Following sequencing, one clone containing a 5 bp deletion creating a premature stop codon (PSC) was selected for SCNT, and six D42 fetuses generated. Western blot of cardiac extracts demonstrated the loss of IL2RG protein. The ILR2RG null PFFs were then used to modify the RAG2 locus by use of CRISPR-Cas9 and cell lines with both homozygous and heterozygous mutations were identified.

The RAG2 mutation frequencies were 80% with 50% being monoallelic mutations and 30% being biallelic. Following sequencing, cell lines carrying loss of function deletions were used for SCNT to generate IL2RG/RAG2 DKO piglets. For allogeneic engraftment donors and recipients were SLA typed for SLA-1, SLA-2, SLA-3, DRB1, DRBQ1 and DQA to ensure they were MHC mismatched. Using SLA-typed HSCs derived from the H2B-GFP line, three IL2RG/RAG2 DKO fetuses underwent fetal injection into the portal system at FD42 of gestation. All three showed significant postnatal allogeneic engraftment in multiple lymphoid organs. As shown in FIG. 2, all three pigs had significant T cell and B cell engraftment. Moreover, the use of the H2B-GFP donor cells allowed efficient identification of the host/donor origin of the lymphoid cells. These results demonstrate the ability to successfully do fetal injection in early gestation, to systematically analyze pigs after transplantation of H2B-GFP labeled cells, and facilitate examining gene editing effects in human cells in the context of a large animal model.

### Example 3

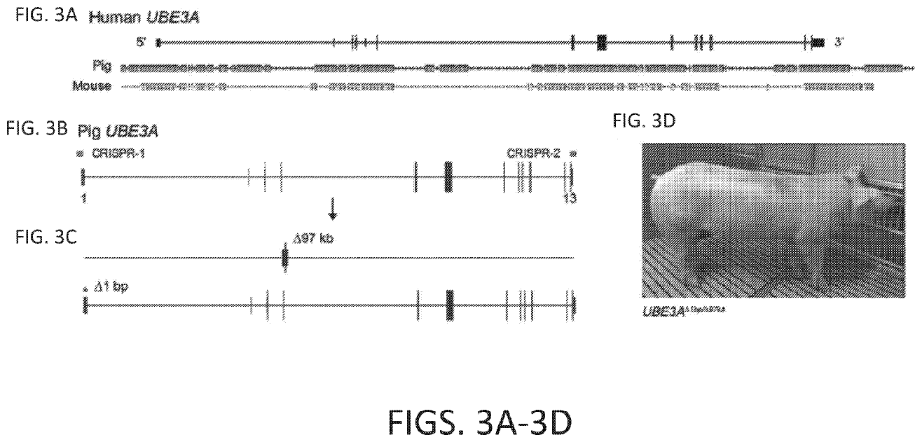

Generation of a Pig Model of Angelman Syndrome (AS). Loss of the maternally inherited ubiquitin E3A ligase (UBE3A) gene causes Angelman syndrome (AS), a devastating neurological disorder characterized by intellectual disability, seizures, happy disposition, absent speech, and seizures. The UBE3A gene is imprinted with maternal-specific expression in the brain and biallelically expressed in all other cell types. Consequently, mutations affecting the maternal UBE3A allele cause AS, whereas mutations affecting the paternal allele are non-penetrant. Currently, there is no effective therapy to treat AS patients. A pig was generated that has mutations in silent paternal UB3A allele (FIG. 3). Males containing a small or a large mutation in UBE3A were generated and sent for breeding and characterization. This demonstrated the ability to generate complex transgenic pigs using the systems and methods of the present disclosure, and provides a way to test different approaches to reactivate the silent, but normal, paternal allele.

In addition, the 1 bp deletion will allow for testing of correction of the expressed maternal allele. This includes CRISPR-based genome and epigenome editors. While not developed as an epigenome reporter per se, this model could be very valuable as a proof of principle that CRISPR-based transcriptional activators can be used for targeted de-repression of silent imprinted genes or correction of indels in the brain.

### Example 4

Measuring Genome-Wide OFF-Target Effects. A key question that will need to be investigated more fully is the frequency and type of in vivo OFF-target effects caused by existing gene editors, as well as newly developed editor and delivery methods. To accomplish this, it is critical that pig-specific baseline data is generated that can be used. At present, there are multiple techniques for examining OTE. Three of the most commonly used and accepted are GUIDE-seq (Tsai et al., 2014), CIRCLE-seq (Tsai et al., 2017) and Digenome-seq (D. Kim et al., 2015). While Digenome-seq looks at genome-wide OTE, it requires a large number of reads (approx. 400 million) and is affected by high background or random DNA reads. The main difference between GUIDE-seq and CIRCLE-seq is that GUIDE-seq requires HDR to insert the tag and as a result has lower sensitivity. This has been resolved in CIRCLEseq and thus was used as part of the methods described herein. Key preliminary data relevant to the embodiments described herein include use of known targets from the human FANCF site 2 (FANCF2), VEGFA site 1 (VEGFA1) and HEK293 site 1 (HEK1) loci. As shown in FIG. 4, comparison of CIRCLE-seq with other approaches demonstrates that CIRCLE-seq has greater sensitivity. Please note that some of the data was generated with selected sites (FANCF2, VEGFA1, and HEK1).

In addition, previous work developed the high fidelity SpCas9-HF1 and used CIRCLEseq data to examine the improvements in efficiency. This work identified OFF-target frequencies when comparing the two enzymes. As shown in FIG. 4, selected targets were part of this analysis and have known frequencies of OTE when using SpCas9-WT or the high fidelity SpCas9-HF1. This difference allows for a) testing the OT reporter by using sites known to cut at high frequencies and b) ensures that those sites are not affected when using the high-fidelity SpCas9.

### Example 5

Experience Using and Developing Adeno Associated Vectors. As part of the validation of the models being generated, it is often advantageous to use a known method of in vivo delivery that can be used to test the reporters. For example, AAVs are used extensively in human clinical trials, are non-pathogenic, and are replication deficient. The following AAV reagents can be used as needed: Spcas9, SaCas9, NmCas9, Cpf1, KRABsaCas9 (nuclease deficient) for transcriptional repression, VP64saCas9 (nuclease def) for transactivation. The following high efficiency AAVs can also be used: AAV9 for systemic multiorgan, AAV2G9 for CNS direct and intraocular, AAV1RX for CNS and cardiac after IV and DETARGETED from liver, AAV2i8 and AAV9.45 for heart and skeletal muscle and DETARGETED from liver, AAV8g9 for liver.

### Example 6

Reporter Constructs and Systems. Each component of the DNA sequence has a purpose, as described further herein, but the combination is a multi-purpose indicator. While removing some of the sequence will impair certain components (e.g., remove the base editor target site and it will not work for base editors but will work for Cre recombinase and nucleases). In some embodiments, the minimum components are the out-of-frame NLS-mCherry or H2B-GFP sequences, the base editor target sites, the nuclease target landing pads/self-tolerizing peptides, the 2a-peptides, the off-target sites. The loxp sites are included for Cre recombinase (to separate delivery from efficiency of the nucleases) but are not necessary to measure nuclease activity. The off-target system and on-target system do not depend upon one another.

It is of note that about ⅓ of NHEJ events will result in the “switching on” of each indicator. This is due to the random nature of DNA repair. The actual frequency can be calculated by scale-up, but the system gives and accurate representation of distribution and frequency of events regardless. The base editing switch does detect all successful editing events, as does the Cre delivery.

Reagents used include DNA plasmids containing mCherry and H2B-GFP, IDT synthesized gBlocks and oligonucleotide primers, Phusion DNA polymerase (ThermoFisher), Gibson Assembly MasterMix (NEB), T4 DNA ligase (NEB), various restriction endonucleases (NEB), Kanamycin and standard E. coli competent cell (NEB5alpha) culture conditions (LB, LB Agar, made in house). Porcine fetal fibroblasts (primary line) were used to test the constructs and for integration of the construct. Nucleofector Amaxa (Lonza) was used to transfect the cells with the DNA constructs.

For genomic integration, reagents include CRISPR/SpCas9 (Addgene #72247) and gRNA (Addgene #43861) plasmids to elicit double-stranded breaks in the genome (in addition to the above reagents) to induce homology directed repair for integration.

On-target effects can be detected for SpCas9, SaCas9, Cpf1, C2c1, TALE, and zinc finger nucleases, in addition to all future programmable nucleases that contain yet-unknown PAM sequences or recognition sites within the “landing pad.” Furthermore, all base editors that elicit a C→T (G→A) or a T→C (A→G) substitution, or nucleases paired with single-strand oligonucleotides to induce small substitutions. Furthermore, frequency of insertion of large genes by HDR or homology independent integration can be detected with the delivery of BFP into the target site. Successful delivery of Cre recombinase also can be detected using the reporter. Off-target effects can be detected for SpCas9, SaCas9, Cpf1, and C2c1, but the system is most specifically geared toward SpCas9.

The two indicators are integrated into the same genomic “safe harbor” region, separated by approximately 800-1000 base pairs. Both sites contain self-tolerizing peptides for GFP and mCherry. The upstream (5′) portion of the indicator contains the ON-target sites, the loxp site for Cre recombination, and H2B-GFP as the indicator for correct genomic editing. It is dependent on an IRES from a genomic promoter (ACTB). The downstream (3′) portion of the reporter contains the base editing target sites, the off-target frequency gradient, and NLS-mCherry as the indicator for gene editing events. It is dependent on a chicken-beta actin promoter for expression.

The reporter can be used in any mammalian cell line or organism, such as, but not limited to, a human cell, a primate cell, a porcine cell, a murine cell, a mammalian cell, an insect cell, an amphibian cell, an avian cell, or a fish cell. In some embodiments, the transgenic reporter can be generated using a line of commercial swine and/or a non-commercial line (e.g., miniature swine/Yucatan). The pig carries a DNA sequence that does not naturally occur anywhere. The physical characteristics will not be detectable without microscopy or DNA sequencing. However, the targeted tissues of the pig will produce a) a non-fluorescent but self-tolerizing immune peptide constantly in all cells and b) either GFP or mCherry (or BFP) when targeted with editors in that cell. In some embodiments, this reporter can be transfected or incorporated into the DNA of any other mammals such as mice, rats, rabbits, or primates.

Reagents include gBlocks containing the sequence of self-tolerizing peptides for either GFP or mCherry, the 2a, and the on- or off-target sites (IDT). These were then assembled into plasmids containing either H2B-GFP or mCherry. (Minor changes were made using digestion and ligation of annealed oligos or site-directed mutagenesis). Regions of homology were amplified from porcine genomic DNA and ligated into plasmids for the construction of the homology directed repair template. Upon final assembly of the HDR template, porcine fetal fibroblasts will be transfected using an endonuclease (SpCas9) in conjunction with the HDR template targeting the porcine ACTB region. Single-cell derived colonies can then be screened for the correct genomic insertion of the construct. These cells will then be used for somatic cell nuclear transfer (SCNT). In future models, the construct can be integrated into the target locus by using homologous recombination to eliminate the need for CRISPRs when generating the model. Once founders are established, the reporter animals will be produced by breeding.

Reagents used include DNA plasmids containing mCherry and H2B-GFP, IDT synthesized gBlocks and oligonucleotide primers, Phusion DNA polymerase (ThermoFisher), Gibson Assembly MasterMix (NEB), T4 DNA ligase (NEB), various restriction endonucleases (NEB), Kanamycin and standard E. coli competent cell (NEB5alpha) culture conditions (LB, LB Agar, made in house). Porcine fetal fibroblasts (primary line) were used to test the constructs and will be used for integration of the construct. Nucleofector Amaxa (Lonza) was used to transfect the cells with the DNA constructs. Porcine fetal fibroblasts the carry the correct insertion of the reporter will be used for somatic cell nuclear transfer.

In vitro, the reporter can be delivered by plasmid (or in the future will be integrated into the cell line). Editors in the form of plasmid, protein or ribo-nucleoprotein complexes can be co-transfected with the plasmid by nucleofection or other transfection reagents. While the plasmid-based reporters were used extensively to develop the systems, the system itself may be most valuable/novel when it is integrated into the DNA of animals.

To generate animal models, the synthetic DNA construct is integrated into genomic DNA. This is either done by a) homology directed repair by using a site-specific nuclease or b) conventional homologous recombination. Once a founding line is established from somatic cell nuclear transfer, the animals will be born with the reporter system integrated into their DNA (e.g., a synthetic gene), and can be bred to generate additional transgenic lines.

For SpCas9, SaCas9, C2c1, and Cpf1: gRNA designed to target the well characterized FANCF site 2, Vegf site 1, or HEK sgRNA1 (Tsai et al. 2014) is delivered to the cells in conjunction with the editor of interest. The same target sites as above will be used for detecting NHEJ vs HDR or insertion: HDR or homology independent insertion constructs can be designed so that the 2a-BFP sequence is in frame with the start codon. Green cells will indicate successful targeting of the cell with an NHEJ outcome while blue events will indicate successful HDR.

On-target effects can be detected for SpCas9, SaCas9, Cpf1, C2c1, TALE, and zinc finger nucleases, in addition to all future programmable nucleases that contain yet-unknown PAM sequences or recognition sites within the “landing pad.” Furthermore, all base editors that elicit a C→T (G→A) or a T→C (A→G) substitution, or nucleases paired with single-strand oligonucleotides to induce small substitutions. Furthermore, frequency of insertion of large genes by HDR or homology independent integration can be detected with the delivery of BFP into the target site. Successful delivery of Cre recombinase also can be detected using the reporter. Off-target effects can be detected for SpCas9, SaCas9, Cpf1, and C2c1, but the off-target system is most specifically geared toward SpCas9.

The fluorescence of ON-target and OFF-target effects can be measured by any standard methods of mCherry or GFP detection. This includes microscopy, flow cytometry, and fluorescence activated cell sorting. Further studies can use DNA sequencing, PCR, and restriction fragment length polymorphism to detect editing. In some embodiments, on target nuclease NHEJ detection: H2B-GFP; off-target nuclease NHEJ detection (for SpCas9, SaCas9, Cpf1, and C2c1): NLS-mCherry; both on and off-target NHEJ events: yellow; Cre-delivery: H2B-GFP; base-editing: NLS-mCherry; and homologous recombination or Homology independent targeted integration (with 2a-BFP template): BFP.

Original design included an FMDV IRES (instead of EMCV IRES-as in the current model), to allow for a gap between the IRES and the start codon. This was intended to allow the base editor target sites to be included in the 5′ (H2B-GFP) switch. However, the FMDV IRES resulted in constant “On” position of the H2B-GFP and therefore the base editing target sites were moved into the chicken beta actin “exon 1” following the chicken beta actin promoter in the 3′ (NLS-mCherry) switch, where it is now functional.

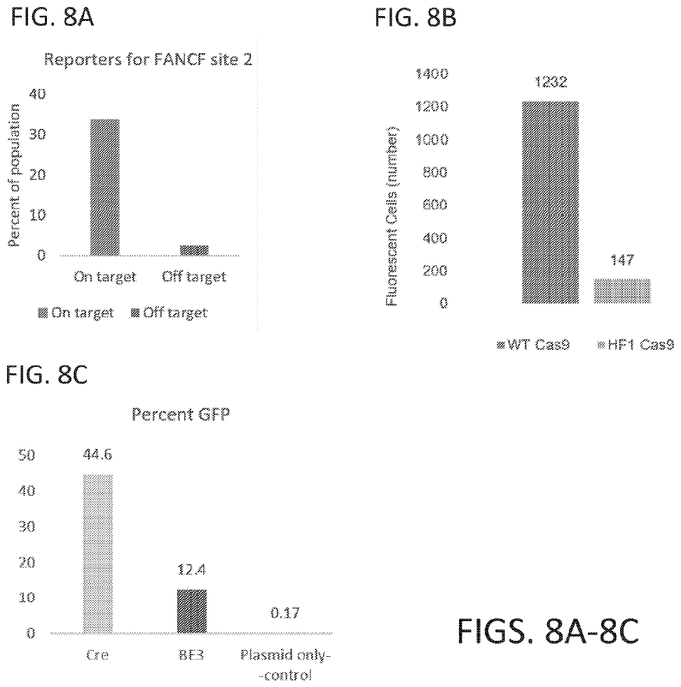

The base-editing switch and the off-target switch were first validated (to accommodate use of flow cytometry machines that detect only GFP and not mCherry) by making several changes to the (pnabio.com/products/Reporter.htm) pHRS (Hygro-gfp) vector. These changes included addition of target sites before the original start codon, alteration of the reading frame before the 2a peptide, and editing of target sites. Once these systems were verified in these plasmids (FIGS. 8A-8C), the base editor target and the off-target sites were incorporated into gBlock sequences (synthesized by IDT). None of the PNA bio plasmid sequence was used in the final design.

In one embodiment, the expression of NLS-mCherry was evaluated when an OFF-target indicator was co-transfected with a CRISPR plasmid and gRNA targeting an off-target site for FANCF2 (FIG. 9). Without a FANCF2-off target specific guide, no cells express NLS-mCherry (data not shown), indicating that the OFF-target construct works properly and remains in an “off” position until DNA is cut and causes a frameshift to shift it to an “on” position and initiate NLS-mCherry expression

Reagents include gRNA targeting the FANCF and leukodystrophy sites cloned into MLM 3636 (Addgene #43861). These were co-transfected with one of the following: High-fidelity SpCas9 (Addgene #72247), Wild-type SpCas9 (Addgene #42230), Base-Editor 3 (Addgene #73021) or Cre recombinase plasmid.

### Example 7

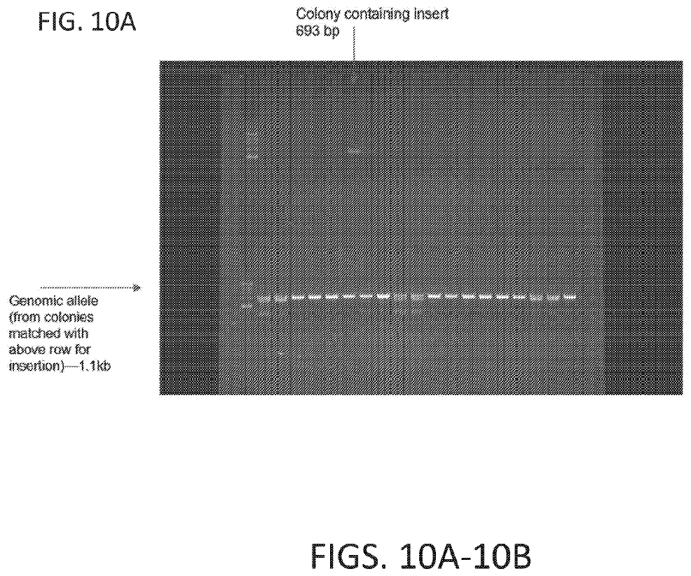

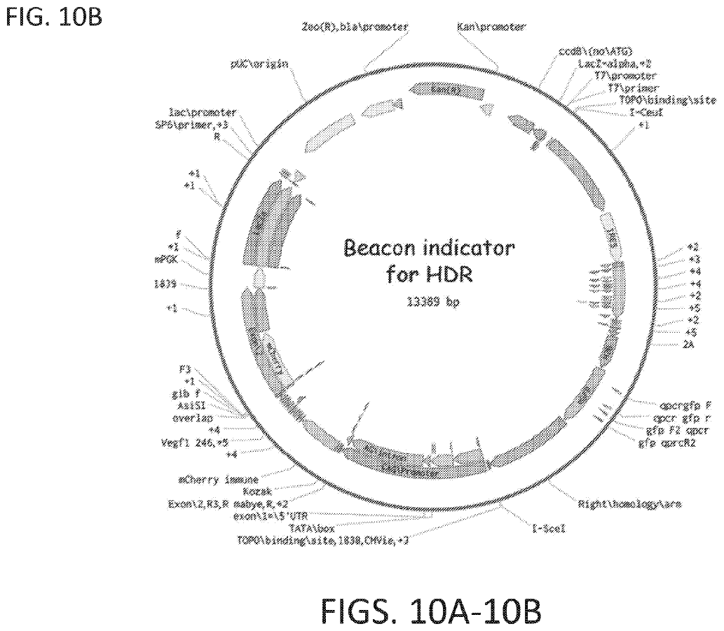

Pig Reporter For Developing and Testing Gene Editing Technologies in a Large Animal Model. Embodiments of the present disclosure include generating a model for use as a lineage/clonal tracer. As shown in FIG. 10A, indicator constructs described herein were inserted into porcine genomic DNA. PCR gels demonstrate genomic insertion of the construct into cellular DNA of porcine fetal fibroblasts. The expected band size is 693 bp (top row). Forward DNA primer is anchored in 3′ end of the transgene insert and reverse primer is anchored in downstream genomic DNA. The approximate frequency for transgene insertion was 6/72 colonies containing the insert. Bottom row shows genomic allele as a control. (See also, FIG. 10B.)

One of the key questions that can be addressed with gene editors as described herein is the negative short-mid and long-term effects. For example, one of these effects could be transformation of a normal cell into a cancerous cell as an unexpected result of the gene editing. There are several characteristics of the constructs provided herein that allow for the determination as to whether a negative event originated out of a gene editing event or was independent from it:

(1) Indels are random in nature. Thus, when a single cell is acted upon by the gene editing enzyme and the reporter is turned on a unique tag will be formed in the reporter. The number of unique “tags” generated by such randomness will be low. Likely less than 100.

(2) The same indels will occur in the Off-target sites. These frequencies will be much lower (rare event) but the number of different “tags” in this region of the construct will also be less than a 100.

(3) In addition, the editors will cleave at very low frequencies other regions of the genome. Those OT sites would have been identified previously and can then be used to identify additional indels in both linked (same chromosome) and unlinked (other chromosomes) sites. In some cases, test gRNAs may have as many as 20-30 OTE sites depending on the design of the guide and the enzyme being used, and each site may have the same frequency of random indels (e.g., 100). 101371 The combination of the three “tags” types then creates a unique and rare tag (frequency 1×frequency 2×frequency 3). This unique tag can then be used to recognize clonal expansion. That is if a gene editing event leads to transformation and tumor formation it will be possible to analyze that tumor and determine if it originated from a single event caused by the gene editing event. Similarly, the same method could be used for cell lineage tracking by again looking how the different tags are segregating as the cells differentiate into a particular pathway. Thus, the reporter constructs described herein can be used to examine clonal expansion as well as to lineage trace cells that have been edited.

What is claimed is:

1. A nucleic acid reporter construct for evaluating functionality of a gene editing system, the construct comprising:
a first reporter cassette comprising:
a first in-frame non-functional fluorescent reporter comprising at least one self-tolerizing peptide and at least one unknown gene editing target site;
a known gene editing target site from at least one human gene; and
a first out-of-frame functional fluorescent reporter; and
a second reporter cassette comprising:
a base editor region comprising at least one base editor target site;
a second in-frame non-functional fluorescent reporter comprising at least one self-tolerizing peptide;
an off-target array region comprising a known gene editing target site from at least one human gene; and
a second out-of-frame functional fluorescent reporter;
wherein the first and second reporter cassettes detect efficiency of a gene editing system based on fluorescence of the at least one first or second out-of-frame functional fluorescent reporter.

2. The reporter construct of claim 1, wherein the first or second in-frame non-functional fluorescent reporter is GFP, mCherry, or BFP.

3. The reporter construct of claim 1 or claim 2, wherein the at least one self-tolerizing peptide comprises an antigenic peptide from a GFP fluorescent reporter, an mCherry fluorescent reporter, or a BFP fluorescent reporter.

4. The reporter construct of any one of claims 1 to 3, wherein the at least one unknown gene editing target site comprises a putative PAM sequence.

5. The reporter construct of claim 4, wherein the putative PAM sequence comprises one or more of NGG, NAG, NGGAG, and TTTN.

6. The reporter construct of any of claims 1 to 5, wherein the known gene editing target site from at least one human gene comprises at least one CRISPR target site from a FANCF gene, a VEGFA gene, a HEK site, a HEK1 intronic site 1, a HEK3 site, a HEK4 site, an EMX gene, or an RNF gene.

7. The reporter construct of claim 6, wherein the known gene editing target site from at least one human gene comprises a plurality of on-target and off-target gene editor target sites.

8. The reporter construct of claim 6, wherein the known gene editing target site from at least one human gene comprises at least one binding site for a CRISPR associated protein.

9. The reporter construct of any one of claims 1 to 8, wherein the first or second out-of-frame functional fluorescent reporter is GFP, mCherry, or BFP.

10. The reporter construct of any one of claims 1 to 9, wherein the first or second out-of-frame functional fluorescent reporter is nuclear localized.

11. The reporter construct of claim 9, wherein the first or second out-of-frame functional fluorescent reporter comprises a 2A peptide sequence.

12. The reporter construct of any one of claims 1 to 11, wherein the at least one base editor target site in the base editor region comprises at least one of an adenine base editor (ABE) or a cytosine base editor (CBE).

13. The reporter construct of claim 12, wherein editing of the at least one base editor target site produces a new proximal ATG site and allows for expression of the second out-of-frame functional fluorescent reporter.

14. The reporter construct of any one of claims 1 to 13, wherein the known gene editing target site from the at least one human gene in the off-target array region comprises at least one CRISPR target site from a FANCF gene, a VEGFA gene, a HEK site, a HEK1 intronic site 1, a HEK3 site, a HEK4 site, an EMX gene, or an RNF gene.

15. The reporter construct of claim 14, wherein the known gene editing target site from the at least one human gene in the off-target array region comprises a plurality of on-target and off-target gene editor target sites.

16. The reporter construct of claim 14, wherein the known gene editing target site from the at least one human gene in the off-target array region comprises at least one binding site for a gene editor associated protein.

17. A cell comprising the reporter construct of any of claims 1 to 16.

18. The cell of claim 17, wherein the cell is one or more of a human cell, a primate cell, a porcine cell, a murine cell, a mammalian cell, an insect cell, an amphibian cell, an avian cell, or a fish cell.

19. A transgenic organism comprising the reporter construct of any one of claims 1 to 16.

20. The transgenic organism of claim 19, wherein the transgenic organism is porcine.

21. A method of assessing functionality of a gene editing system, the method comprising:
subjecting a transgenic organism comprising the reporter construct of any of claims 1 to 16 to a gene editing system; and
detecting fluorescence of the at least one first and/or second out-of-frame functional fluorescent reporter.

22. A nucleic acid reporter construct for evaluating functionality of a gene editing system, the construct comprising:
a reporter cassette comprising:
a base editor region comprising at least one base editor target site;
an in-frame non-functional fluorescent reporter comprising at least one self-tolerizing peptide; and
an out-of-frame functional fluorescent reporter;
wherein the reporter cassette detects efficiency of a gene editing system based on fluorescence of the at least one out-of-frame functional fluorescent reporter.

23. The reporter construct of claim 22, wherein the in-frame non-functional fluorescent reporter is GFP, mCherry, or BFP.

24. The reporter construct of claim 22 or claim 23, wherein the at least one self-tolerizing peptide comprises an antigenic peptide from a GFP fluorescent reporter, an mCherry fluorescent reporter, or a BFP fluorescent reporter.

25. The reporter construct of any one of claims 22 to 24, wherein the out-of-frame functional fluorescent reporter is nuclear localized.

26. The reporter construct of any of claims 22 to 25, wherein the at least one base editor target site in the base editor region comprises at least one of an adenine base editor (ABE) or a cytosine base editor (CBE).

27. The reporter construct of claim 26, wherein editing of the at least one base editor target site produces a new proximal ATG site and allows for expression of the second out-of-frame functional fluorescent reporter.

28. A nucleic acid reporter construct for evaluating functionality of a gene delivery system, the construct comprising:
a first reporter cassette comprising:
a first in-frame non-functional fluorescent reporter comprising at least one self-tolerizing peptide and at least one unknown gene editing target site;
a known gene editing target site from at least one human gene; and
a first out-of-frame functional fluorescent reporter; and
a second reporter cassette comprising:
a base editor region comprising at least one base editor target site;
a second in-frame non-functional fluorescent reporter comprising at least one self-tolerizing peptide;
an off-target array region comprising a known gene editing target site from at least one human gene; and
a second out-of-frame functional fluorescent reporter;
wherein the first and second reporter cassettes detect efficiency of a gene delivery system based on fluorescence of the at least one first or second out-of-frame functional fluorescent reporter.
